(19) United States
(12) Patent Application Publication
(10) Pub. No.: US 2010/0331597 A1
(43) Pub. Date: Dec. 30, 2010

(54) Catalyst And Once-Through Reactor-Regenerator Process For Oxygenate To Olefins Production

(76) Inventors: Guang Cao, Princeton, NJ (US); Chunshe Cao, Houston, TX (US); Richard B. Hall, SanFord, FL (US); Thomas H. Colle, Houston, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 12/779,414

(22) Filed: May 13, 2010

Related U.S. Application Data
(60) Provisional application No. 61/221,383, filed on Jun. 29, 2009.

(51) Int. Cl. C07C 1/00

(52) U.S. Cl. 585/640

(57) ABSTRACT

Disclosed herein is a method of converting oxygenates to olefins comprising contacting an oxygenate stream with an acidic high silica chabazite catalyst in one or more oxygenate-to-olefins reactors; circulating greater than from 80% of the catalyst to one or more catalyst regenerators to form regenerated catalyst; circulating the regenerated catalyst, preferably the same amount of regenerated catalyst, back to the oxygenate-to-olefins reactor to contact an oxygenate stream; and isolating a stream of olefins from the one or more oxygenate-to-olefins reactors.

### CROSS-REFERENCE TO RELATED APPLICATION

This application claims priority to U.S. Provisional Patent Application No. 61/221,383, filed Jun. 29, 2009, the disclosure of which is incorporated herein by reference in its entirety.

### FIELD OF THE INVENTION

The disclosure herein relates to the use of a high-silica chabazite molecular sieve catalyst in an oxygenate-to-olefins conversion process in a once-through reactor system where a large portion, or all, of the catalyst is regenerated after reacting with oxygenate.

### BACKGROUND OF THE INVENTION

Molecular sieves are useful catalysts in the conversion of methanol to olefins, or more generally, the conversion of oxygenates to olefins (or “OTO”). The “high chabazite SAPO” class of catalysts demonstrates a characteristic induction period in which its activity for methanol conversion and selectivity for prime olefins increase with initial contacting with methanol (and hence, generation of coke in the pores of the molecular sieve catalyst). The lower selectivity associated with this induction period is mitigated with such process design as disclosed in, for example, US 2004-0105787, incorporated by reference, which allows a fraction of the coked catalyst to enter the regenerator while the majority is recycled back to the riser without regeneration. This way of operation generates a catalyst mixture that has a statistically distributed amount of coke on the molecular sieve catalyst particles. However, the resulting better prime olefins selectivity is accompanied by a larger catalyst inventory and lower average catalyst activity due to a certain amount of coke on catalyst.

In contrast, fluid catalyst cracking (“FCC”) reactors runs in a once-through mode in which all catalyst leaving the riser reactor and the stripper is regenerated in the regenerator to burn off coke. Coke on the regenerated catalyst that returns to the riser/reactor is low, typically below 1%. The operation is simple and the average catalyst activity is higher than fractional regeneration due to lower coke on catalyst entering the reaction zone.

While a once-through reactor/regenerator system has operational and design advantages, it is only a good option if a catalyst has little or none of the selectivity disadvantages of the above-mentioned SAPOs having an induction period. The inventors have found that the induction period is essentially absent in high-silica chabazite OTO catalyst, especially at higher temperatures. The inventors find that high-silica chabazite is a good fit with the advantageous FCC-like once-through process for oxygenate conversion to light olefins, and perhaps other types of catalysis.

Some publications of interest include US 2003-0176751 A1, U.S. Pat. No. 7,008,610 B2, U.S. Pat. No. 7,067,108 B2, US 2007-0100185 A1, U.S. Pat. No. 7,094,389 B2, US 2007-0287874 A1, US 2007-0286798 A1, US 2008-0103345 A1, and “Synthesis and structure of pure SiO₂ chabazite, the SiO₂ polymorph with the lowest framework density” in CHEM. COMMUN. 1881 (1998), each of which is incorporated by reference.

### SUMMARY OF THE INVENTION

Disclosed herein is a method of converting oxygenates to olefins comprising contacting an oxygenate stream with an acidic high silica chabazite catalyst in one or more oxygenate-to-olefins reactors; circulating greater than from 80% (by total weight of catalyst (molecular sieve, binder, etc.) contacted with oxygenate) of the catalyst to one or more catalyst regenerators to form regenerated catalyst; circulating the regenerated catalyst back to the oxygenate-to-olefins reactor to contact an oxygenate stream; and isolating a stream of olefins from the one or more oxygenate-to-olefins reactors.

In certain embodiments, the oxygenate stream comprises a mixture of fresh oxygenate and recycled oxygenate.

In certain other embodiments, substantially all of the acidic high silica chabazite catalyst is circulated to the catalyst regenerator.

In certain other embodiments, the average residence time of the acidic high silica chabazite catalyst in the catalyst regenerator is within the range from 1 to 30 min.

In certain other embodiments, the average catalyst regenerator temperature is within the range from 200 to 1500° C.

In certain other embodiments, the average coke level of the acidic high silica chabazite catalyst in the reactor/regenerator system, preferably after regeneration and before contacting with the oxygenate, is less than from 5 wt % by weight of the catalyst.

In certain other embodiments, the temperature of the acidic high silica chabazite catalyst is maintained at greater than from 200° C. throughout the contacting and regeneration process and pathways there between.

In certain other embodiments, the one or more oxygenate-to-olefins reactors are riser reactors.

In certain other embodiments, the silica-to-aluminum ratio of the acidic high silica chabazite catalyst is within the range from 10 to 2000.

The various descriptive elements and numerical ranges disclosed herein can be combined with other descriptive elements and numerical ranges to describe preferred embodiments of the apparatus and process described herein; further, any upper numerical limit of an element can be combined with any lower numerical limit of the same element to describe preferred embodiments. In this regard, the phrase “within the range from X to Y” is intended to include within that range the “X” and “Y” values.

### BRIEF DESCRIPTION OF THE DRAWINGS

FIG. 1 is a graphical representation of Prime Olefins Selectivity (“POS”, ethylene plus propylene selectivity) as a function of the cumulative grams of methanol converted per gram of sieve (“CMCPS”) in the methanol-to-olefins conversion using the comparative (open symbols) and inventive (closed symbols) catalysts at 25 psig and a WHSV of 100 g MeOH/g sieve/hr;

FIG. 2 is a graphical representation of Methanol Conversion as a function of the CMCPS in the methanol-to-olefins conversion using the comparative (open symbols) and inventive (closed symbols) catalysts at 25 psig and a WHSV of 100 g MeOH/g sieve/hr;

FIG. 3 is a graphical representation of Prime Olefins Selectivity in the methanol-to-olefins conversion as a function of the CMCPS at a desirable reaction temperature using the comparative (open symbols) and inventive (closed symbols) catalysts at 25 psig and a WHSV of 100 g MeOH/g sieve/hr;

FIG. 4 is a graphical representation of Methanol Conversion in the methanol-to-olefins conversion process as a function of the CMCPS at a desirable reaction temperature using the comparative (open symbols) and inventive (closed symbols) catalysts at 25 psig and a WHSV of 100 g MeOH/g sieve/hr; and

FIG. 5 is a side-view of one possible embodiment of a once-through type of reactor/regenerator system that would be useful in the process described herein.

### DETAILED DESCRIPTION

The disclosure herein relates to a reactor system for converting oxygenates to olefins, especially ethylene and propylene, using a molecular sieve catalyst that functions best when regenerated upon each cycle of contacting the molecular sieve with oxygenate. In one aspect, described herein is a method of converting oxygenates to olefins comprising, or consisting essentially of in a particular embodiment, contacting an oxygenate stream with an acidic high silica chabazite catalyst (“HiSi-CHA”) in one or more oxygenate-to-olefins reactors; circulating greater than from 80 or 85 or 90 or 95 or 99% (by total weight of catalyst (molecular sieve, binder, etc.) contacted with oxygenate) of the catalyst upon each cycle of contacting with oxygenate to one or more catalyst regenerators to form regenerated catalyst; circulating the same amount of regenerated HiSi-CHA back to the oxygenate-to-olefins reactor to contact an oxygenate stream; and isolating a stream of olefins from the one or more oxygenate-to-olefins reactors. In a particular embodiment, substantially all of the HiSi-CHA is circulated to the catalyst regenerator upon each cycle of contacting with oxygenate. By “substantially all” what is meant is that the regenerator and oxygenate-to-olefins reactor is set up to circulate all (an amount of catalyst equal to the amount reacted with oxygenate) of the catalyst through the at least one regenerator. Also, in the context above, “consisting essentially of means the process does not include any other regeneration and/or reaction steps or stages, but may include minor features well known in the art such as pumps, vents, compressors, heaters, coolers, filters, etc.

As used herein, the term “regenerated catalyst” is a catalyst that has been exposed to regeneration conditions in the catalyst regenerator, an apparatus described further herein and based on common designs known in the art. The term “regenerated catalyst” applies to catalyst that has passed through the catalyst regenerator at regeneration conditions and not necessarily to catalyst with a specific loading of coke. However, the average loading of coke in the catalyst, regenerated and oxygenate-contacted, is maintained at a level of less than from 5 or 4 or 3 or 2 or 1 or 0.1 wt % by weight of the catalyst in certain embodiments. Stated another way, the steady state level of coke on the catalyst is desirable less than from 5 or 4 or 3 or 2 or 1 or 0.1 wt % by weight of the catalyst.

By this manner, greater than from 80 or 85 or 90 or 95 or 99%, or substantially all, of the molecular sieve catalyst that is brought into contact with oxygenate has been treated as by regeneration of the catalyst to reduce the coke level as described herein, and the same amount or all of that regenerated catalyst is then recirculated to contact oxygenate. This process is preferably substantially continuous or completely continuous, meaning that it does not stop on a regular basis. In certain embodiments, greater than from 80 or 85 or 90 or 95 or 99%, or substantially all, of the molecular sieve catalyst that is brought into contact with oxygenate has been treated as by regeneration of the catalyst, including supplemental fresh catalyst that has not been circulated in the reactor/regenerator system. “Fresh” catalyst is molecular sieve catalyst that has less than from 5 or 4 or 3 or 2 or 1 or 0.1 wt %, by weight of the catalyst, of coke either due to it being freshly synthesized, or because it has been regenerated ex situ from another location and added to the reactor/regenerator system described herein. The fresh catalyst, when present, may comprise less than from 1 or 5 or 10 or 15 or 20 wt %, by weight of all the molecular sieve catalyst in the system. In certain embodiments, when fresh catalyst is added to the system, some, or an equivalent amount, of catalyst in the system is withdrawn.

As used herein, the term “acidic high silica chabazite catalyst” or “HiSi-CHA” refers to solid particles of desirable size comprising molecular sieve having a chabazite structure as described by D. W. Breck in ZEOLITE MOLECULAR SIEVES (John Wiley & Sons, 1973) and comprising a silica-to-aluminum ratio greater than from 10 or 20 or 30 or 60 or 100 or 200 in certain embodiments; or described another way, within the range from 10 or 20 or 30 or 60 or 100 or 150 or 200 to 250 or 300 or 350 or 400 or 450 or 500 or 1000 or 2000 in certain embodiments. The desirable HiSi-CHA is formed using a bulky organoamine hydroxide or fluoride directing agent. A “bulky” agent is one that fills the space of a cyclohexane molecule or larger. The desirable HiSi-CHA is described further below.

### Acidic High Silica Chabazite Catalyst

The desirable HiSi-CHA used herein is a form of chabazite, preferably manufactured in a fluoride- and/or hydroxide-containing medium. In its calcined form, the useful HiSi-CHA has an X-ray diffraction pattern having the characteristic lines shown in Table 1 below:

TABLE 1
Typical HiSi-CHA Diffraction Pattern

| d(Å) | Relative Intensities (I %) |
|---|---|
| 9.36-8.98 | 80-100 |
| 6.86-6.66 | 20-60 |
| 6.33-6.15 | 0-10 |
| 5.51-5.38 | 5-40 |
| 4.97-4.86 | 5-50 |
| 4.63-4.54 | 0-10 |
| 4.28-4.20 | 20-60 |
| 3.94-3.87 | 0-10 |
| 3.83-3.76 | 0-10 |
| 3.54-3.49 | 5-40 |
| 3.41-3.36 | 5-40 |
| 3.14-3.10 | 0-10 |
| 2.889-2.853 | 5-50 |
| 2.850-2.815 | 5-40 |
| 2.650-2.620 | 0-10 |
| 2.570-2.542 | 0-10 |
| 2.467-2.441 | 0-10 |
| 2.244-2.223 | 0-10 |
| 2.088-2.070 | 0-10 |
| 2.059-2.041 | 0-10 |
| 1.883-1.869 | 0-10 |
| 1.842-1.828 | 0-10 |

These X-ray diffraction data were collected with a Siemens powder X-Ray Diffractometer, equipped with a scintillation detector with graphite monochromator, using copper K-alpha radiation. The diffraction data were recorded by step-scanning at 0.02 degrees of two-theta, where theta is the Bragg angle, and a counting time of 1 second for each step. The interplanar spacing, d's, were calculated in Angstrom units, and the relative intensities of the lines, I/Iₒ is one-hundredth of the intensity of the strongest line, above background were determined by integrating the peak intensities. The intensities are uncorrected for Lorentz and polarization effects. It should be understood that diffraction data listed for this sample as single lines may consist of multiple overlapping lines which under certain conditions, such as differences in crystallographic changes, may appear as resolved or partially resolved lines. Typically, crystallographic changes can include minor changes in unit cell parameters and/or a change in crystal symmetry, without a change in the framework atom connectivities. These minor effects, including changes in relative intensities, can also occur as a result of differences in cation content, framework composition, nature and degree of pore filling, crystal size and shape, preferred orientation and thermal and/or hydrothermal history.

The HiSi-CHA described herein has a composition involving the molar relationship (1):

X₂O₃:(n)YO₂, (1)

wherein X is a trivalent element, such as aluminum, boron, iron, indium, and/or gallium, typically aluminum; Y is a tetravalent element, such as silicon, tin, titanium and/or germanium, typically silicon; and “n” is greater than from 10 or 20 or 30 or 60 or 100 or 200 in certain embodiments; or described another way, within the range from 10 or 20 or 30 or 60 or 100 or 150 or 200 to 250 or 300 or 350 or 400 or 450 or 500 or 1000 or 2000 in certain embodiments.

In its as-synthesized form, the HiSi-CHA useful herein has a composition involving the molar relationship (2):

X₂O₃:(n)YO₂:(m)R:(x)F:(z)H₂O, (2)

wherein X, Y and “n” are as defined in the preceding paragraph and wherein “m” ranges from 0.01 to 2, such as from 0.1 to 1, “z” ranges from 0.5 to 100, and “x” is a value within the range from 0 to 2 in certain embodiments. In particular embodiments, the number of aluminum atoms per chabazite unit cell of the HiSi-CHA is within the range from 0.1 or 0.25 or 0.5 to 1.2 or 1.6 or 1.8 or 2. In certain embodiments, the acidic high silica chabazite catalyst is substantially free from phosphorous atoms. By “substantially free,” what is meant is that phosphorous containing compounds are not added to the synthesis mixture and are kept out of the synthesis mixture in forming the crystallized molecular sieve, but may be present only as a minor impurity in a reactant, such as by less than from 0.01 wt % of the composition.

The HiSi-CHA useful herein can be prepared from a reaction mixture containing sources of water, an oxide of a trivalent element (X), an oxide of a tetravalent element (Y), an organic directing agent (R) as described below, and fluoride ions (F), the reaction mixture having a composition, in terms of mole ratios of oxides, within the following ranges:

| Reactants | Useful | Typical |
|---|---|---|
| H₂O/YO₂ | 2-40 | 2-5 |
| F/YO₂ | 0.2-1.0 | 0.4-0.8 |
| R/YO₂ | 0.2-2.0 | 0.3-1.0 |
| X₂O₃/YO₂ | 0.00025-0.02 | 0.0005-0.01 |

The organic directing agent R used herein is conveniently selected from bulky organoamine hydroxides or bulky organoamine fluorides in certain embodiments. In a more particular embodiment, the bulky organoamine hydroxide or bulky organoamine fluoride directing agent is selected from the hydroxide or fluoride salts of N,N,N—C1 to C10 alkyl substituted piperidines, N,N,N—C1 to C10 alkyl substituted cyclohexylammoniums, N,N,N—C1 to C10 alkyl substituted adamantylammoniums and N,N,N—C1 to C10 alkyl substituted aminonorbornanes, and mixtures thereof. The “C1 to C10 substitution” is referring to straight or branched alkyls bound to the nitrogen atom of the compound. The base hydrocarbon atoms (i.e., the cyclohexyl, adamantyl, etc.) may also be C1 to C10 and/or halogen substituted. In yet a more particular embodiment, the bulky organoamine hydroxide or bulky organoamine fluoride directing agent is selected from the hydroxide or fluoride salts of N,N,N—C1 to C10 alkyl substituted cyclohexylammoniums, and mixtures thereof.

In certain embodiments, the water, a source of fluoride ions (e.g., the organic directing agent and/or HF, etc.), when used, and sources of silicon and alumina are combined at a temperature within the range from 100 or 120 or 140 to 200 or 220 or 260° C. to form the acidic high silica chabazite catalyst. These ingredients are preferably combined while the solution is stirred.

Crystallization of the catalyst can be carried out at either static or stirred conditions in a suitable reactor vessel, such as for example, polypropylene jars or Teflon®-lined or stainless steel autoclaves, at a temperature of 100 or 120 or 140° C. to 200 or 225° C. for a time sufficient for crystallization to occur at the temperature used, for example, from 16 hours to 7 days. Synthesis of the new crystals may be facilitated by the presence of at least 0.01% seed crystals, based on total weight of the crystalline product, in one embodiment, and at least 0.10% in a more particular embodiment, and at least 1% in yet a more particular embodiment.

After crystallization is complete, the crystals are separated from the mother liquor, washed and calcined to remove the organic directing agent. Calcination is typically conducted at a temperature within the range from 370° C. to 925° C. for at least 1 minute and generally not longer than 20 hours. If needed, additional activation of the HiSi-CHA can be effected, such as by cation exchange or acidification techniques.

As in the case of many catalysts, it may be desirable to incorporate the resultant chabazite with another material resistant to the temperatures and other conditions employed in organic conversion processes. Such materials include catalytically active and inactive materials and synthetic or naturally occurring zeolites as well as inorganic materials such as clays, silica and/or metal oxides such as alumina The latter may be either naturally occurring or in the form of gelatinous precipitates or gels including mixtures of silica and metal oxides. Use of a catalytically active material tends to change the conversion and/or selectivity of the catalyst in the oxygenate conversion process. Inactive materials suitably serve as diluents to control the amount of conversion in the process so that products can be obtained in an economic and orderly manner without employing other means for controlling the rate of reaction. These materials may be incorporated into naturally occurring clays, for example, bentonite and kaolin, to improve the crush strength of the catalyst under commercial operating conditions. Such materials, that is, clays, oxides, etc., function as binders for the catalyst. It is desirable to provide a catalyst having good crush strength because in commercial use it is desirable to prevent the catalyst from breaking down into powder-like materials.

Naturally occurring clays which can be employed include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays can be used in the raw state as originally mined or initially subjected to calcination, acid treatment or chemical modification. Other useful binders include inorganic oxides, such as silica, zirconia, titania, magnesia, beryllia, alumina, and mixtures thereof.

In addition to the foregoing materials, the HiSi-CHA can be composited with a porous matrix material such as silica-alumina, silica-magnesia, silica-zirconia, silica-thoria, silica-beryllia and silica-titania as well as ternary compositions such as silica-alumina-thoria, silica-alumina-zirconia, silica-alumina-magnesia and silica-magnesia-zirconia.

The relative proportions of HiSi-CHA and inorganic oxide matrix may vary widely, with the zeolite content ranging from 1 to 90 percent by weight and more usually, particularly when the composite is prepared in the form of beads, in the range of 2 or 4 or 8 or 10 or 20 to 60 or 70 or 80 wt %, by weight of the catalyst composition.

### OTO and Regeneration

The HiSi-CHA described herein is particularly suitable for use in a process for converting organic oxygenates to olefins rich in ethylene and propylene. As used herein, the term “oxygenates” is defined to include, but is not necessarily limited to aliphatic alcohols, ethers, carbonyl compounds (aldehydes, ketones, carboxylic acids, carbonates, and the like), and also compounds containing hetero-atoms, such as, halides, mercaptans, sulfides, amines, and mixtures thereof. The aliphatic moiety will normally contain from 1 to 10 carbon atoms, such as from 1 to 4 carbon atoms. Representative oxygenates include lower straight chain or branched aliphatic alcohols, their unsaturated counterparts, and their nitrogen, halogen and sulfur analogues. Examples of suitable oxygenate compounds include methanol; ethanol; n-propanol; isopropanol; alcohols; methyl ethyl ether; dimethyl ether; diethyl ether; di-isopropyl ether; methyl mercaptan; methyl sulfide; methyl amine; ethyl mercaptan; diethyl sulfide; diethyl amine; ethyl chloride; formaldehyde; dimethyl carbonate; dimethyl ketone; acetic acid; n-alkyl amines, n-alkyl halides, n-alkyl sulfides; and mixtures thereof. Particularly suitable oxygenate compounds are methanol, dimethyl ether, or mixtures thereof, most preferably methanol. As used herein, the term “oxygenate” designates only the reactive material used as the feed. The total charge of feed to the reaction zone may contain additional compounds, such as diluents.

In the present oxygenate conversion process, a feedstock comprising an organic oxygenate, optionally with a diluent, is contacted in the vapor phase in a reaction zone with a catalyst comprising the HiSi-CHA at effective process conditions so as to produce the desired olefins. Alternatively, the process may be carried out in a liquid or a mixed vapor/liquid phase. When the process is carried out in the liquid phase or a mixed vapor/liquid phase, different conversion rates and selectivities of feedstock-to-product may result depending upon the catalyst and the reaction conditions. In certain embodiments, the oxygenate stream comprises a mixture of fresh oxygenate and recycled oxygenate.

To convert oxygenate to olefin, any variety of reactor systems can be used, including fixed bed, fluid bed, or moving bed systems. In a particular embodiment, the oxygenate-to-olefins reactor(s) is a co-current riser reactor(s) and short contact time, countercurrent free-fall reactor(s). Fixed beds are generally not preferred for the process because oxygenate to olefin conversion is a highly exothermic process which requires several stages with intercoolers or other cooling devices. The reaction also results in a high pressure drop due to the production of low pressure, low density gas. Each OTO “reactor” may comprise one, two, three, four, five or more riser reactors or “risers,” typically fluidly connected to a central zone to collect the spent catalyst. The spent catalyst can then be partially or entirely portioned to one or more catalyst regenerators. An example of such a system is described in, for example, US 2004-0105787 and U.S. Pat. No. 7,083,762, incorporated by reference.

The temperature employed in the OTO process may vary over a wide range. Although not limited to a particular temperature, best results will be obtained if the process is conducted at temperatures within the range from 200 or 300 or 350 or 400 or 450 to 550 or 600 or 650 or 700° C.

Light olefin products will form, although not necessarily in optimum amounts, at a wide range of pressures, including but not limited to autogeneous pressures and pressures within the range from 0.1 kPa to 100 MPa. In other embodiments, the pressure is in the range of from 6.9 kPa to 1000 kPa, and within the range from 48 kPa to 340 kPa in a yet a more particular embodiment. The foregoing pressures are exclusive of diluent, if any is present, and refer to the partial pressure of the feedstock as it relates to oxygenate compounds and/or mixtures thereof.

The process should be continued for a period of time sufficient to produce the desired olefin products. The reaction time may vary from tenths of seconds to a number of hours. The reaction time is largely determined by the reaction temperature, the pressure, the catalyst selected, the weight hourly space velocity, the phase (liquid or vapor) and the selected process design characteristics.

A wide range of weight hourly space velocities (“WHSV”) for the feedstock will function in the present process. WHSV is defined as weight of feed (excluding diluent) per hour per weight of a total reaction volume of molecular sieve catalyst (excluding inerts and/or fillers). In certain embodiments, the WHSV in the oxygenate-to-olefins reactor is greater than from 1 or 10 or 20 or 40 or 60 or 80 grams methanol/grams catalyst/hour. In yet more particular embodiments, the WHSV in the oxygenate-to-olefins reactor is within the range from 1 or 10 or 20 or 40 or 60 or 80 to 120 or 140 or 160 or 180 grams methanol/grams catalyst/hour.

One or more diluents may be fed to the reaction zone with the oxygenates, such that the total feed mixture comprises diluent in a range of from 1 mol % to 99 mol %. Diluents which may be employed in the process include, but are not necessarily limited to, helium, argon, nitrogen, carbon monoxide, carbon dioxide, hydrogen, water, paraffins, other hydrocarbons (such as methane), aromatic compounds, and mixtures thereof. Typical diluents are water and nitrogen.

Ideally, most or all of the coke should be removed from most or all of the HiSi-CHA prior to each contacting of the oxygenate in the OTO reactor. Because the catalyst must be regenerated frequently, the reactor should allow easy removal of at least portion to all of the catalyst to a regenerator, where the catalyst is subjected to a regeneration medium, such as a gas comprising oxygen, for example air, to burn off coke from the catalyst, which restores the catalyst activity. Preferably, there is a continuous flow of catalyst through the reactor/regenerator system. The conditions of temperature, oxygen partial pressure, and residence time in the regenerator should be selected to achieve a coke content on regenerated catalyst of less than from 5 or 4 or 3 or 2 or 1 or 0.5 or 0.1 wt %. At least a portion of the regenerated catalyst should be returned to the reactor, in certain embodiments greater than from 80 or 85 or 90 or 95 or 99% of the catalyst, and all of the catalyst in a particular embodiment, is circulated to one or more catalyst regenerators to form regenerated catalyst.

In certain embodiments, the average residence time of the HiSi-CHA in the catalyst regenerator is within the range from 1 or 2 or 5 to 7 or 10 or 12 or 20 or 30 min; and in other embodiments the average residence time is within the range from 5 or 10 or 15 to 20 or 30 min. In certain embodiments, the average catalyst regenerator temperature is within the range from 200 or 300 or 450 or 550 to 750 or 1000 or 1500° C. In yet other embodiments, the temperature of the HiSi-CHA is maintained at greater than from 200 or 300 or 400 or 450° C. throughout the olefin contacting and regeneration process and pathways there between. The regeneration and OTO reaction conditions should be such that the initial prime olefins selectivity is greater than from 60 or 65 or 70 or 75 wt %, by weight of the olefin reaction product from the oxygenate-to-olefins reaction.

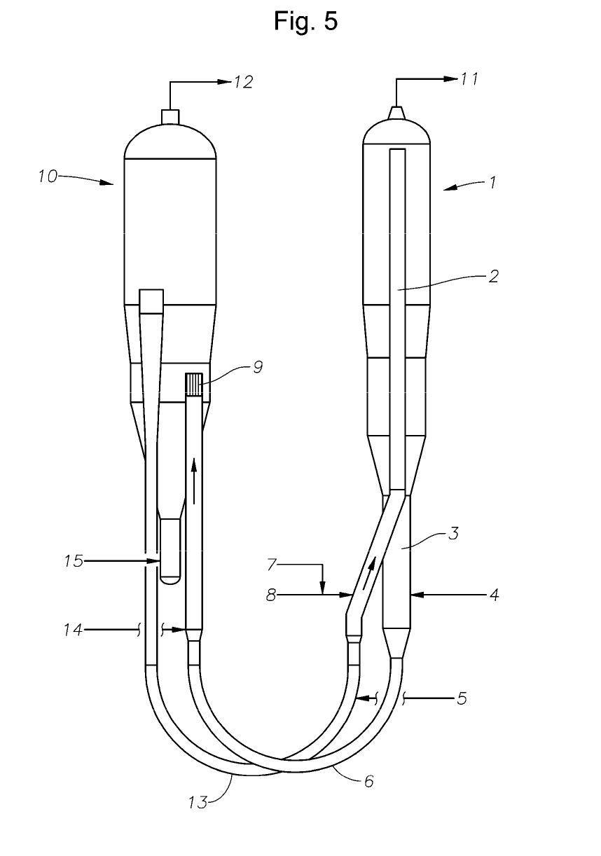

An embodiment of the OTO reactor/catalyst regenerator system is described with respect to FIG. 5. In that embodiment, the OTO reactor 1 is fluidly connected to the catalyst regenerator 10 through at least the spent catalyst transfer means 6 and the regenerated catalyst transfer means 13. Both of these transfer means are insulated and/or temperature controlled to achieve and/or maintain a certain desirable catalyst temperature. The molecular sieve catalyst is “spent” when it has contacted the fresh/recycle oxygenate through the riser reactor and is then separated from the olefin product to be directed to the catalyst regenerator. Fresh catalyst, if desired is injected into the OTO reactor at stream 5, and fresh oxygenate stream 7 and/or recirculated oxygenate stream 8 is then contacted with the catalyst in the lower portion of the riser 2 to form olefin product which is removed from the reactor through stream 11. There is an optional stripping section 3 of the OTO reactor where catalyst can be subjected to steam treatment such as described in US 2007-0286798, incorporated by reference.

The spent catalyst then makes its way down the lower portion 4 of the reactor 1 through the spent catalyst transfer means 6 to the spent catalyst intake portion 14 of the regenerator 10 where the catalyst is deposited through intake 9 into the regenerator. The regenerator may have any desirable configuration as known in the art and may include a fluidized bed of catalyst within. The regenerator oxidation medium, such as air or pure oxygen or mixture of oxygen with another gas, enters the regenerator as stream 15 in the described embodiment in order to contact the spent catalyst and create regenerated molecular sieve catalyst, that is, catalyst having less than from 5 or 4 or 3 or 2 or 1 or 0.1 wt % coke present within the catalyst. In any case, the indicated amount (as a percentage of catalyst) is regenerated and sent out of the regenerator 10 through the regenerated catalyst transfer means 13 back to the reactor 1. In desirable embodiments, the same amount, or an amount within 2 or 5 or 10 or 20 wt % of the total catalyst within both transfer means, is transferred to and from the reactor 1 through the transfer means 6 and 13. Regenerator exhaust such as, for example, carbon dioxide is released in stream 12. As described, at least some or all of the catalyst is continuously circulated through the entire OTO reactor 1 and regenerator 10 system. This arrangement desirably allows for a low to no catalyst inventory to be maintained within the system.

The ethylene and/or propylene produced by the OTO methods herein can be used to make any number of ethylene-based and/or propylene-based polymers. It is well known in the art to contact the ethylene and/or propylene with any number of polyolefin polymerization catalysts such as titanium based Ziegler-Natta catalysts or Group 4-based metallocene catalysts or chromium catalysts, and others. Polymers such as polypropylene or polyethylene or copolymers thereof can be isolated therefrom.

The examples described below are non-limiting demonstrations of the features of the inventions) described herein.

### Examples

The catalysts tested were a comparative SAPO-34 catalyst and a HiSi-CHA made by the method described generally in US 2003-0176751, and more particularly below. The comparative sample was a typical SAPO-34 AEI/CHA intergrowth having higher CHA character.

### Synthesis of Example HiSi-CHA

A 23.5 g/liter aqueous solution of Al(NO₃)₃.9H₂O (23.9 ml) was added to a 0.66 M aqueous solution (336.2 ml) of N,N,N-dimethylethylcyclohexylammonium hydroxide (“DMECHA”). To the solution was further added 100 ml (92.5 g) tetraethylorthosilicate. The mixture was sealed in a polypropylene bottle and shaken for about 72 hours at room temperature for tetraethylorthosilicate to completely hydrolyze. To the clear solution obtained was added 48 wt % aqueous solution of hydrofluoric acid (11.58 g), which resulted in an immediate precipitation. This mixture slurry was made uniform by vigorous shaking and was poured into a plastic dish for evaporation of water and ethanol at room temperature. A stream of nitrogen was directed toward the mixture to facilitate solvent evaporation. Immediately prior to the end of this process, 2.9 g of colloidal LEV seeds (containing 11 wt % LEV or “levyne,” Si/Al=6) was added. The evaporation step was terminated once the weight of the mixture reached 172.0 g. The nearly dry solid had the following composition:

0.5(DMECHA⁺OH⁻):0.6HF:1.0SO₂:(1/400)Al₂O₃:4.0H₂O.

The resulting mixture was transferred to eight Teflon lined 23 ml autoclaves and was heated at 180° C. for 65 hours while being tumbled (40 rpm). The solid product was recovered by repeated centrifuging and washing with distilled water, and finally drying in a 50° C. vacuum oven. 29.4 g product was recovered and confirmed to be pure chabazite by XRD. Elemental analysis indicates the sample has Si/Al ratio of 223.

The methanol-to-olefins (“MTO”) reaction used to test embodiments of the described herein was carried out on a fixed-bed microreactor, and during the test methanol was fed at a preset pressure and flow rate to a stainless steel reactor tube housed in an isothermally heated zone. The reactor tube contained about 10-50 mg as-synthesized molecular sieve catalyst mixed with about 200 mg SiC. The catalyst had been calcined (ramp to 650° C. and hold for up to three hours in air) before being loaded to the reactor tube, and was activated for 30 minutes at 500° C. in flowing helium before methanol was admitted. The product effluent was sampled, at different times during the run, with a twelve-port sampling loop while the catalyst was continuously deactivating. The effluent sample in each port was analyzed with a Gas Chromatograph equipped with an FID detector.

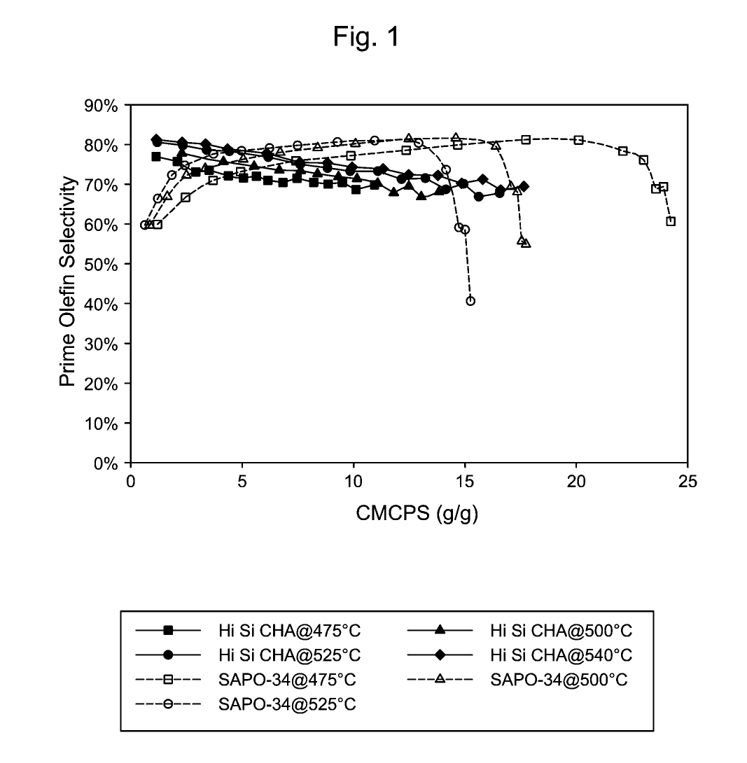

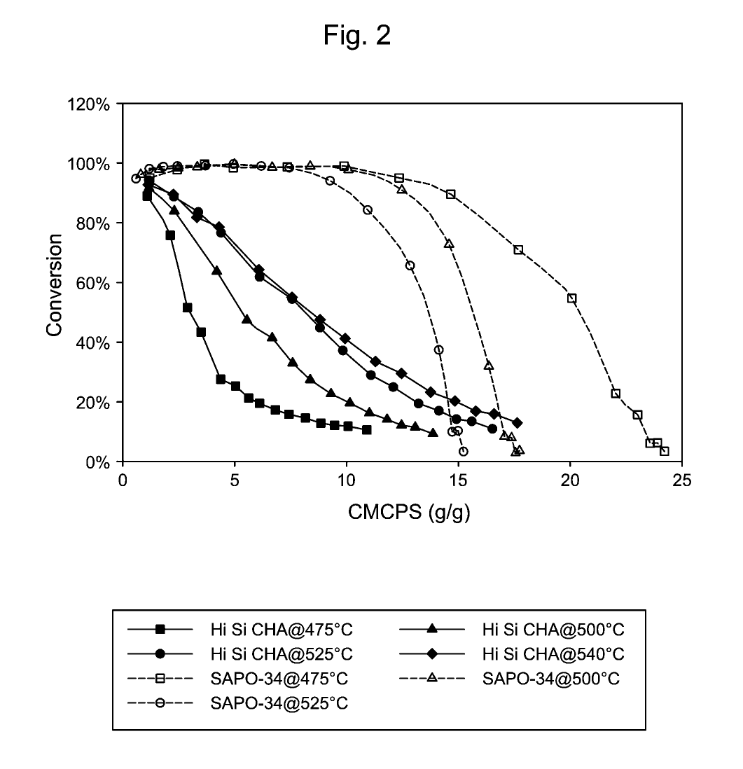

The testing conditions were as follows: the temperature in the microreactor was varied from 475 to 525° C. for SAPO-34 and 475 to 540° C. for the HiSi-CHA; the pressure of methanol was 40 psia. The feed rate expressed as weight hourly space velocity (“WHSV”) was 100 g MeOH/g sieve/hr. Cumulative conversion of methanol was expressed as cumulative grams of methanol converted per gram of sieve (“CMCPS”). On-stream lifetime refers to the CMCPS when methanol conversion has dropped to 10%. The product selectivity was reported as averages over the entire conversion range, rather than from a single point in effluent composition. The testing results are shown in FIGS. 1 and 2, where the filled symbols and solid lines represent HiSi-CHA performance while the open symbols and dotted lines represent SAPO-34 data.

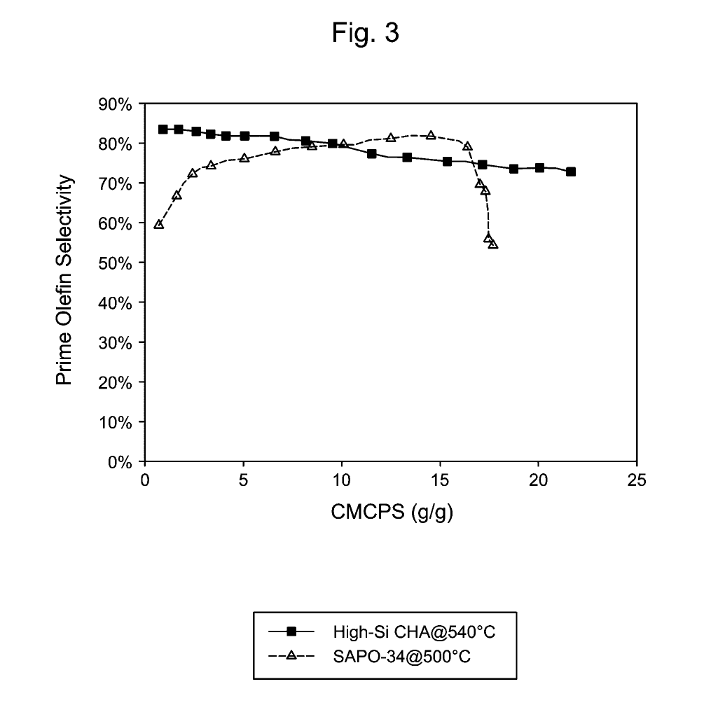

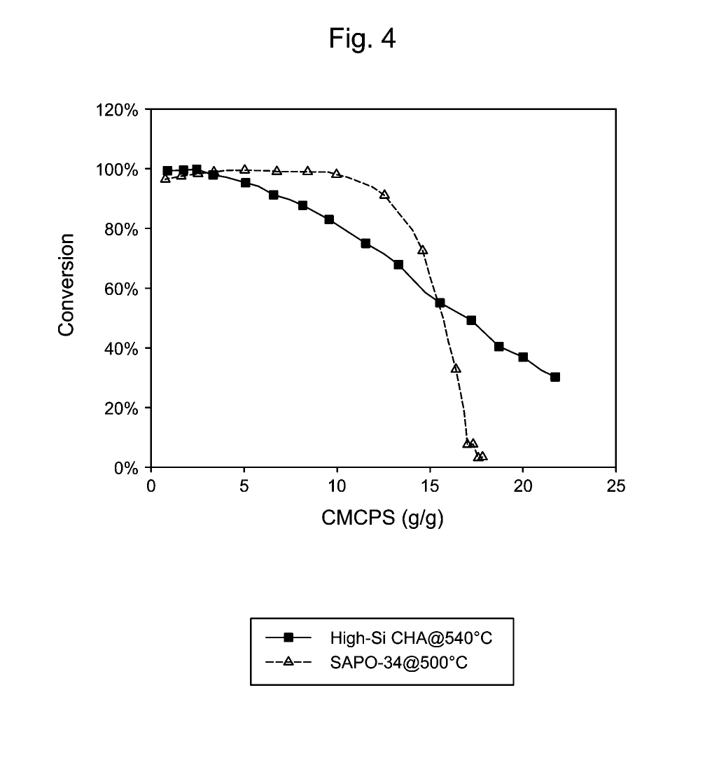

A pair-wise comparison of the key performance characteristics is shown in FIGS. 3 and 4.

The results show that the prime olefin selectivity (“POS”, ethylene plus propylene selectivity) is the highest at the earliest time on stream (lowest CMCPS), and decreases with increasing CMCPS, whereas the SAPO-34 catalyst goes through the typical induction period at the same temperature. Therefore advantage can be taken of this initial high selectivity by running a once-through, FCC-like process for better selectivity. The conversion data (FIG. 2) shows that HiSi-CHA (Si/Al=223) has little induction in activity as well, attaining high activity at very low CMCPS. It is also shown that higher temperature reaction enhances prime olefin selectivity for HiSi-CHA, and reduces coking deactivation rate which in turn increases on-stream lifetime.

Table 2 below summaries the initial and average performance of both SAPO-34 and the HiSi-CHA. The average prime olefin selectivity is the conversion weighted average integrated from 0 to 10 grams of MeOH converted per gram of sieve. It can be seen that SAPO-34 catalyst does not reach its peak conversion and maximum prime olefins selectivity at initial on-oil time, while HiSi-CHA achieves the highest activity and selectivity on fresh catalysts (initial performance=peak performance). The integrated (average) selectivity for prime olefin production out to 10 g/g CMCPS is significantly higher for the HiSi-CHA. Once-through reactor-regenerator design with full coke burn mode would maintain a minimum coke level on the catalysts, so that the maximum performance (up to 95% MeOH conversion and 81% prime olefins selectivity) can be potentially achieved.

TABLE 2
Pressure of 25 psig, WHSV of 100 g MeOH/g sieve/hour

| | | | | Initial Prime | |
|---|---|---|---|---|---|
| | | | Peak | Olefins | Average Prime Olefin |
| T | | Initial MeOH | MeOH | Selectivity, | Selectivity from |
| (° C.) | Catalyst | conversion, % | conversion, % | wt % | 0 to 10 g CMCPS, wt % |
| 475 | SAPO-34 | 95.1 | 99.7 | 60 | 71 |
| | HiSi-CHA | 89.2 | 89.2 | 76.9 | 74 |
| 500 | SAPO-34 | 96.4 | 99.7 | 59.9 | 73 |
| | HiSi-CHA | 92.5 | 92.5 | 79 | 75 |
| 525 | SAPO-34 | 95 | 99.8 | 59.8 | 75 |
| | HiSi-CHA | 94.3 | 94.3 | 80.7 | 77 |
| 540 | HiSi-CHA | 92.8 | 92.8 | 81.2 | 80 |

Having described the process and the apparatus and its various features, described herein in numbered embodiments is:
1. A method of converting oxygenates to olefins comprising, or consisting essentially of in a particular embodiment, the following steps:
   contacting an oxygenate stream with an acidic high silica chabazite catalyst in one or more oxygenate-to-olefins reactors;
   circulating greater than from 80% of the catalyst upon each cycle of contacting with oxygenate to one or more catalyst regenerators to form regenerated catalyst;
   circulating the regenerated catalyst back to the oxygenate-to-olefins reactor to contact an oxygenate stream; and
   isolating a stream of olefins from the one or more oxygenate-to-olefins reactors.
2. The method of numbered embodiment 1, wherein the oxygenate stream comprises a mixture of fresh oxygenate and recycled oxygenate.
3. The method of numbered embodiments 1 and 2, wherein substantially all of the acidic high silica chabazite catalyst is circulated to the catalyst regenerator.
4. The method any one of the previously numbered embodiments, wherein the average residence time of the acidic high silica chabazite catalyst in the catalyst regenerator is within the range from 1 to 30 min.
5. The method any one of the previously numbered embodiments, wherein the average catalyst regenerator temperature is within the range from 200 to 1200° C.
6. The method any one of the previously numbered embodiments, wherein the average coke level of the acidic high silica chabazite catalyst in the reactor/regenerator system, preferably after regeneration and before contacting with the oxygenate, is less than from 5 wt % by weight of the catalyst.
7. The method any one of the previously numbered embodiments, wherein the temperature of the acidic high silica chabazite catalyst is maintained at greater than from 200° C. throughout the contacting and regeneration process and pathways there between.
8. The method any one of the previously numbered embodiments, wherein the one or more oxygenate-to-olefins reactors comprise riser reactors.
9. The method any one of the previously numbered embodiments, wherein the silica-to-aluminum ratio of the acidic high silica chabazite catalyst is greater than from 10.
10. The method any one of the previously numbered embodiments, wherein the silica-to-aluminum ratio of the acidic high silica chabazite catalyst is within the range from 10 to 2000.
11. The method any one of the previously numbered embodiments, wherein the oxygenate-to-olefins reactor temperature is within the range from 200 to 700° C.
12. The method any one of the previously numbered embodiments, wherein the initial prime olefins selectivity is greater than from 60 wt %, by weight of the olefin reaction product from the oxygenate-to-olefins reaction.
13. The method any one of the previously numbered embodiments, wherein the WHSV in the oxygenate-to-olefins reactor is greater than from 1 grams methanol/grams catalyst/hour.
14. The method any one of the previously numbered embodiments, wherein the WHSV in the oxygenate-to-olefins reactor is within the range from 1 to 180 grams methanol/grams catalyst/hour.
15. The method any one of the previously numbered embodiments, wherein the acidic high silica chabazite catalyst is produced using a bulky organoamine hydroxide or bulky organoamine fluoride directing agent.
16. The method numbered embodiment 15, wherein the bulky organoamine hydroxide or bulky organoamine fluoride directing agent is selected from the hydroxide or fluoride salts of N,N,N—C1 to C10 alkyl substituted piperidines, N,N,N—C1 to C10 alkyl substituted cyclohexylammoniums, N,N,N—C1 to C10 alkyl substituted adamantylammoniums and N,N,N—C1 to C10 alkyl substituted aminonorbornanes, and mixtures thereof.
17. The method of numbered embodiment 15, wherein the bulky organoamine hydroxide or bulky organoamine fluoride directing agent is selected from the hydroxide or fluoride salts of N,N,N—C1 to C10 alkyl substituted cyclohexylammoniums, and mixtures thereof.
18. The method any one of the previously numbered embodiments, wherein water, a source of fluoride ions and sources of silicon and alumina are combined at a temperature within the range from 100 to 260° C. to form the acidic high silica chabazite catalyst.
19. The method any one of the previously numbered embodiments, wherein the number of aluminum atoms per chabazite unit cell of the acidic high silica chabazite catalyst is within the range from 0.1 to 2.
20. The method any one of the previously numbered embodiments, wherein the acidic high silica chabazite catalyst is substantially free from phosphorous atoms.
21. The method of any one of the previously numbered embodiments, wherein ethylene and/or propylene is isolated from the olefins stream and contacted with a polymerization catalyst to form a polyolefin.

Also described herein is the use of at least one riser reactor in fluid connection with at least one catalyst regenerator to contact an oxygenate stream with an acidic high silica chabazite catalyst in one or more of the oxygenate-to-olefins reactors; circulate greater than from 80% of the catalyst upon each cycle of contacting with oxygenate to one or more of the catalyst regenerators to form regenerated catalyst; circulate the regenerated catalyst back to the oxygenate-to-olefins reactor(s) to contact an oxygenate stream; and isolate a stream of olefins from the one or more oxygenate-to-olefins reactors.

What is claimed is:

1. A method of converting oxygenates to olefins comprising:
contacting an oxygenate stream with an acidic high silica chabazite catalyst in one or more oxygenate-to-olefins reactors;
circulating greater than from 80% of the catalyst upon each cycle of contacting with oxygenate to one or more catalyst regenerators to form regenerated catalyst;
circulating the regenerated catalyst back to the oxygenate-to-olefins reactor(s) to contact an oxygenate stream; and
isolating a stream of olefins from the one or more oxygenate-to-olefins reactors.

2. The method of claim 1, wherein the oxygenate stream comprises a mixture of fresh oxygenate and recycled oxygenate.

3. The method of claim 1, wherein substantially all of the acidic high silica chabazite catalyst is circulated to the catalyst regenerator.

4. The method of claim 1, wherein the average residence time of the acidic high silica chabazite catalyst in the catalyst regenerator is within the range from 1 to 30 min.

5. The method of claim 1, wherein the average catalyst regenerator temperature is within the range from 200 to 1200° C.

6. The method of claim 1, wherein the average coke level of the acidic high silica chabazite catalyst in the reactor/regenerator system is less than from 5 wt % by weight of the catalyst.

7. The method of claim 1, wherein the temperature of the acidic high silica chabazite catalyst is maintained at greater than from 200° C. throughout the contacting and regeneration process and pathways there between.

8. The method of claim 1, wherein the one or more oxygenate-to-olefins reactors comprise riser reactors.

9. The method of claim 1, wherein the silica-to-aluminum ratio of the acidic high silica chabazite catalyst is greater than from 10.

10. The method of claim 1, wherein the silica-to-aluminum ratio of the acidic high silica chabazite catalyst is within the range from 10 to 2000.

11. The method of claim 1, wherein the oxygenate-to-olefins reactor temperature is within the range from 200 to 700° C.

12. The method of claim 1, wherein the initial prime olefins selectivity is greater than from 60 wt %, by weight of the olefin reaction product from the oxygenate-to-olefins reaction.

13. The method of claim 1, wherein the WHSV in the oxygenate-to-olefins reactor is greater than from 1 grams methanol/grams catalyst/hour.

14. The method of claim 1, wherein the WHSV in the oxygenate-to-olefins reactor is within the range from 1 to 180 grams methanol/grams catalyst/hour.

15. The method of claim 1, wherein the acidic high silica chabazite catalyst is produced using a bulky organoamine hydroxide or bulky organoamine fluoride directing agent.

16. The method of claim 15, wherein the bulky organoamine hydroxide or bulky organoamine fluoride directing agent is selected from the hydroxide or fluoride salts of N,N,N—C1 to C10 alkyl substituted piperidines, N,N,N—C1 to C10 alkyl substituted cyclohexylammoniums, N,N,N—C1 to C10 alkyl substituted adamantylammoniums and N,N,N—C1 to C10 alkyl substituted aminonorbornanes, and mixtures thereof.

17. The method of claim 15, wherein the bulky organoamine hydroxide or bulky organoamine fluoride directing agent is selected from the hydroxide or fluoride salts of N,N,N—C1 to C10 alkyl substituted cyclohexylammoniums, and mixtures thereof.

18. The method of claim 1, wherein water, a source of fluoride ions and sources of silicon and alumina are combined at a temperature within the range from 100 to 260° C. to form the acidic high silica chabazite catalyst.

19. The method of claim 1, wherein the number of aluminum atoms per chabazite unit cell of the high silica chabazite catalyst is within the range from 0.1 to 2.

20. The method of claim 1, wherein the acidic high silica chabazite catalyst is substantially free from phosphorous atoms.

21. The method of claim 1, wherein ethylene and/or propylene is isolated from the olefins stream and contacted with a polymerization catalyst to form a polyolefin.

22. A method of converting oxygenates to olefins consisting essentially of:
contacting an oxygenate stream with an acidic high silica chabazite catalyst in one or more oxygenate riser reactors;
continuously circulating greater than from 80% of the catalyst upon each cycle of contacting with oxygenate to one or more catalyst regenerators to form regenerated catalyst, wherein the average residence time of the acidic high silica chabazite catalyst in the catalyst regenerator is within the range from 1 to 30 min;
continuously circulating the at least the same amount of regenerated catalyst back to the oxygenate riser reactors to contact an oxygenate stream; and
isolating a stream of olefins from the one or more oxygenate-to-olefins reactors;
wherein the average coke level of the acidic high silica chabazite catalyst is less than from 5 wt % by weight of the catalyst.

23. The method of claim 22, wherein the acidic high silica chabazite catalyst is produced using a bulky organoamine hydroxide or bulky organoamine fluoride directing agent.

24. The method of claim 22, wherein water, a source of fluoride ions and sources of silicon and alumina are combined at a temperature within the range from 100 to 260° C. to form the acidic high silica chabazite catalyst.

25. The method of claim 22, wherein the acidic high silica chabazite catalyst is substantially free from phosphorous atoms.
